(19) United States
(12) Patent Application Publication
(10) Pub. No.: US 2016/0096040 A1
(43) Pub. Date: Apr. 7, 2016

(54) Laminated Needles and Methods of Making and Using Same

(71) Applicant: Tenex Health, Inc., Lake Forest, CA (US)

(72) Inventor: Tate Ray Parham, Silverado, CA (US)

(21) Appl. No.: 14/505,392

(22) Filed: Oct. 2, 2014

(51) Int. Cl. A61N 7/00; A61B 17/32; B23K 31/02; B01F 11/02; B23K 1/00

(52) U.S. Cl. CPC A61N 7/00; B01F 11/0258; B23K 1/0008; B23K 31/02; A61B 17/320068; B01F 2215/0034

(57) ABSTRACT

Laminated needle assemblies and methods of their manufacture and use are provided herein.

### BACKGROUND

The present disclosure is directed to laminated needles for delivering ultrasonic energy as well as to methods for making and using such needles.

### DESCRIPTION OF THE RELATED ART

Needles can experience a number of stresses or forces. Of particular concern are those forces that are non-coaxial to the needle, i.e., the direction of the force is not coaxial with the direction of the needle, thereby causing the needles to bend and eventually break. Unfortunately, the force exerts the greatest influence at the point along the needle where the needle is attached to some support structure whether it be a syringe or the horn of an ultrasonic delivery device.

Also, in some settings, such as when delivering ultrasonic energy using a needle, the ultrasonic energy itself can cause the needle to break irrespective of the outside forces applied to the needle.

These problems are exacerbated as the length of the needle increases. Longer needles are required for some procedures, and increasing the gauge of the needle is not always an option. Knowing this, practitioners accept that the needle will break more often and need replacing more frequently.

Thus, there exists a need for a needle that can withstand greater non-coaxial forces, particular when delivering ultrasonic energy and particularly for applications where a long, narrow gauge needle is desired.

### SUMMARY

Disclosed herein are laminated needle assemblies for delivering ultrasonic energy. The laminated needle assemblies can include a first elongate portion and a second elongate portion. The first elongate portion defines a first length and has an inner diameter, an outer diameter, a proximal end, and a distal end. The second elongate portion defines a second length, is coaxially aligned with the first elongate portion and has an inner diameter, an outer diameter, a proximal end, and a distal end. In some embodiments, the first length of the first elongate portion is greater than the second length of the second elongate portion. In some embodiments, the inner diameter of the second elongate portion is no less than the outer diameter of the first elongate portion, and the second elongate portion is configured to surround at least a portion of an outer surface of the first elongate portion. In some embodiments, the second elongate portion is secured to the first elongate portion at one of or both the distal end and the proximal end of the second elongate portion.

Also disclosed herein are methods of using the laminated needle assemblies disclosed herein to deliver ultrasonic energy. The ultrasonic energy may be delivered to both organic and inorganic material. In some methods, the laminated needle assembly delivers ultrasonic energy to a metal or a plastic. In some methods, the ultrasonic energy is delivered as a part of a medical procedure, such as a urology procedure, plastic surgery, tenotomy, fasciotomy, wound debridement, etc. According to some methods, the laminated needle assemblies disclosed herein are used to deliver ultrasonic energy to mix one or more compounds. In some methods, the mixing is achieved in a difficult-to-access region, which may be a region of the human body. According to some methods, the laminated needle assemblies of the present disclosure are used to deliver ultrasonic energy to machine or shape a material. Such materials can include glass materials, such as quartz. Ultrasonic energy may also be used with the laminated needles disclosed herein in welding applications, such as plastic welding.

Also disclosed herein are methods of manufacturing a laminated needle assembly. The methods can include securing a first elongate portion to a second elongate portion—the first elongate portion defining a first length and comprising: an inner diameter, an outer diameter, a proximal end, and a distal end—the second elongate portion defining a second length and coaxially aligned with the first elongate portion and comprising: an inner diameter, an outer diameter, a proximal end, and a distal end. In some methods, the first length of the first elongate portion is greater than the second length of the second elongate portion. In some methods, the inner diameter of the second elongate portion is no less than the outer diameter of the first elongate portion, the second elongate portion configured to surround at least a portion of an outer surface of the first elongate portion. In some methods, the second elongate portion is secured to the first elongate portion at at least one of the distal end and the proximal end of the second elongate portion.

These and other features are disclosed in greater detail in the accompanying figures and the Detailed Description below.

### BRIEF DESCRIPTION OF THE DRAWINGS

FIG. 1 is a perspective view of an ultrasonic delivery device that includes at its distal end a laminated needle assembly according to some embodiments of the present disclosure.

FIG. 2 is a side view of a laminated needle assembly according to some embodiments of the present disclosure.

FIG. 3 is a cross-sectional view of the laminated needle assembly of FIG. 2 taken along line 3-3.

FIG. 4 is a cross-section view of the laminated needle assembly of FIGS. 2 and 3 taken along line 4-4.

### DETAILED DESCRIPTION

The present disclosure is directed to laminated needle assemblies. Such assemblies may be used in ultrasonic applications or any application where extra needle strength is desirable. Also disclosed herein are methods of manufacturing such needles as well as methods in which the needles may be used. However, the present disclosure is not limited to the precise manufacturing methods and applications disclosed herein. Skilled artisans will recognize that other methods could be employed to achieve similar results or that the disclosed methods could be modified without materially changing the scope of this disclosure.

In ultrasonic applications, there is a need for a needle that can transmit ultrasonic energy while maintaining rigidity and strength. According to some embodiments of laminated needle assemblies disclosed herein, the laminated needle comprises a plurality of needle subunits, each subunit being coaxially aligned with each other. The plurality of needle subunits may be joined by any suitable manner, such as by crimping, welding, or brazing. The needle subunits may be mechanically secured or fastened at both ends to create a parallel beam structure that provides a surprising amount of strength.

Laminating separate needle subunits to each other increases the cross-sectional area of the resulting needle. Without being tied to any particular theory, it is believed that the resulting increase in strength exhibited by the laminated needle as compared to the strength of the first needle subunit alone is roughly proportional to the increase in cross-sectional area. However, rather than simply increasing the cross-sectional area of a single needle, the use of multiple needle submits, each of which is successively shorter, can achieve a similar increase in strength without undesirably increasing the size of the tip or distal end of the needle.

In the context of the present disclosure, the term “laminated” is used in accordance with its ordinary meaning in this field and includes the combination of various components to form a unitary whole, for example, the combination of two or more needle subunits to form a single needle assembly where each subunit fits around or over the preceding subunit and all the subunits are secured to each other to form the unitary assembly.

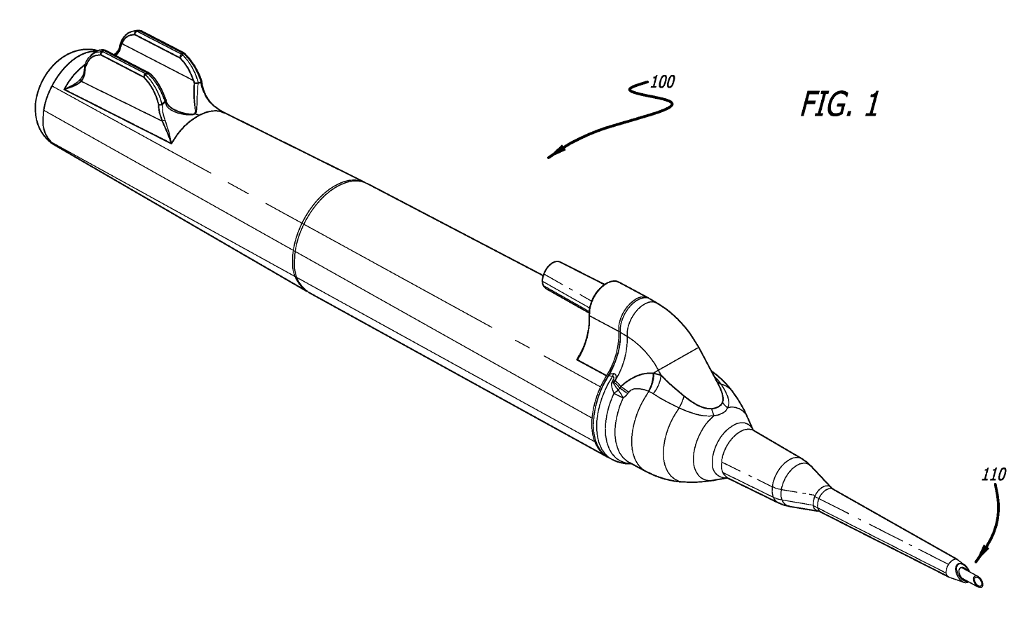

FIG. 1 illustrates a perspective view of ultrasonic delivery device 100 having a proximal end and a distal end with laminated needle assembly 110 located at the distal end of the device. Laminated needle assembly 110 may be used with any suitable ultrasonic delivery device. Laminated needle assembly 110 may be configured to be secured within the horn of ultrasonic delivery device 100, as disclosed in U.S. Publication Nos. 2010/0312102, 2011/0160620, and 2013/0331872. Each of these disclosures, particularly as they relate to ultrasonic delivery devices, is incorporated herein by reference.

According to some embodiments disclosed herein, a laminated needle assembly comprises at least two needle subunits or elongate portions. The first needle is generally longer than the second and subsequent needles. Each needle includes a proximal end and a distal end. In some embodiments, the first needle is positioned inside and coaxially aligned with the second and subsequent needle subunits. Any suitable number of needle subunits may be used in the laminated needles of the present disclosure. Although the addition of each needle subunit adds to the overall size and width of the laminated needle assembly, each needle subunit also adds to the strength of the assembly. The skilled artisan can determine, based on the desired application, how many subunits to include.

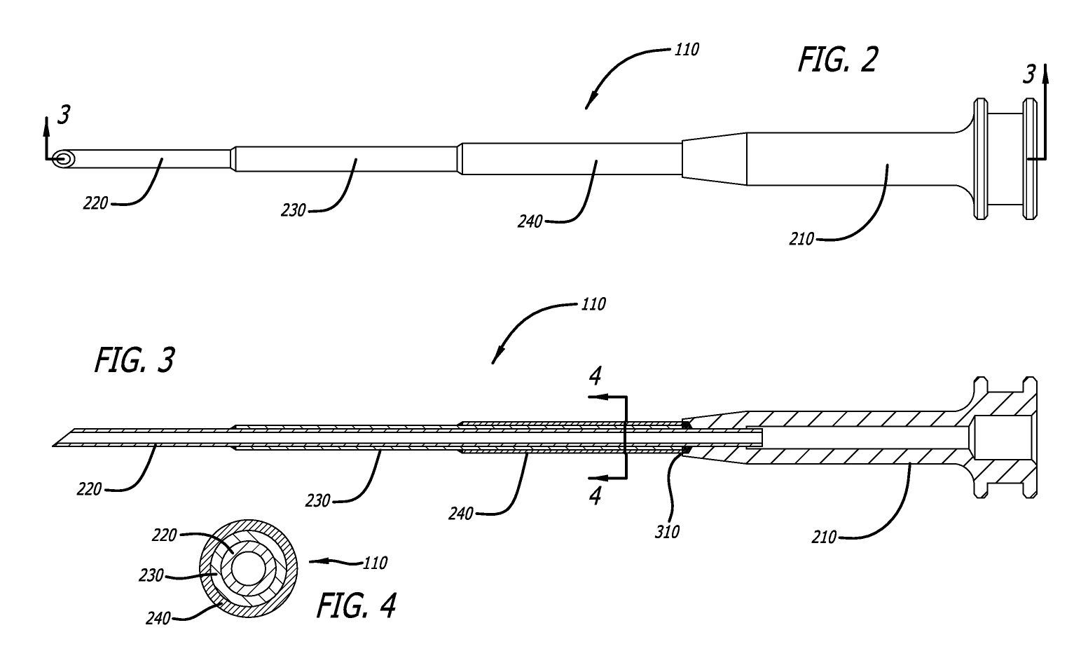

FIG. 2 illustrates an embodiment of laminated needle assembly 110 secured to horn 210. Horn 210 is configured to be secured within the handpiece of an ultrasonic delivery device, such as ultrasonic delivery device 100 shown in FIG. 1. Laminated needle assembly 110 includes three needle subunits or elongate portions: first needle subunit 220, second needle subunit 230, and third needle subunit 240. In this embodiment, each successive needle subunit is shorter than the previous subunit.

Each needle subunit may comprise one or more materials. Suitable materials include metals and non-metals. Suitable metals can include steel, stainless steel, alloys—such as nitinol, amorphous metal alloys (e.g., Liquidmetal and Vitreloy), etc. Suitable non-metals can include ceramics, polymers, etc. Examples of ceramics include aluminum oxide, aluminum nitride, and zirconia (zirconium dioxide), though other ceramics could also be used.

According to some embodiments where stainless steel is used for at least one needle subunit, the stainless steel is chosen from 300 series stainless steels (e.g., 302, 303, 304, 304L, 316, and 317). In some embodiments, a 200 series stainless steel comprises at least a portion of at least one needle subunit.

According to some embodiments, the material comprising at least a portion of at least one needle is a work-hardened material. For example, a stainless steel may be used that has been hardened by work hardening, which can occur during a redraw process. In some cases, it is desirable to avoid the use of an annealing process, which could weaken the material.

In some embodiments, at least one needle subunit comprises a material distinct from the one or more other needle subunits. For example, in some embodiments, first needle subunit 220 comprises stainless steel, and second needle subunit 230 and third needle subunit 240 comprise a metal other than stainless steel. In some embodiments, at least one needle comprises titanium. For example, in some embodiments, titanium is used as the material for the first needle subunit, and a stainless steel is used as the material for the subsequent needle subunit(s). Without being tied to any theory, it is believed that the strength of titanium could be better harnessed if one or more outer needles comprising a stainless steel are used to lend rigidity to the longer, inner titanium needle.

Each needle subunit has a proximal end and a distal end. In some embodiments, each successive needle subunit is secured to the underlying or previous needle subunit at one or both ends. For example, in some embodiments, second needle subunit 230 is secured to first needle subunit 220 at only the proximal end or at only the distal end of second needle subunit 230. In some embodiments, second needle subunit 230 is secured to first needle subunit 220 at both the proximal and the distal end. Similarly, third needle subunit 240—or any subsequent needle subunit—may be secured to second needle subunit 230 at one or both of the proximal end and the distal end of third needle subunit 240. In some embodiments, each subsequent needle subunit may be secured to the previous or underlying needle subunit at a point other than a proximal or a distal end.

FIG. 3 illustrates a cross-section of needle assembly 110 shown in FIG. 2 taken along line 3-3. First needle subunit 220 has a length that is greater than the respective lengths of second needle subunit 230 and third needle subunit 240. Similarly, second needle subunit 230 has a length that is greater than third needle subunit 240. In some embodiments, and as shown in FIG. 3, the proximal ends of second needle subunit 230 and third needle subunit 240 are substantially aligned whereas the proximal end of first needle subunit 220 extends beyond the proximal ends of second needle subunit 230 and third needle subunit 240.

Laminated needle assembly 110 is secured to horn 210 using any suitable process, which can include brazing, welding, crimping, friction fit, etc. In the illustrated embodiment, laminated needle assembly 110 is secured to horn 210 at tip 310 using a brazing process. Tip 310 comprises a recessed portion configured to receive a brazing material. The brazing material contacts horn 210, first needle subunit 220, second needle subunit 230, and third needle subunit 240. In the illustrated embodiment, additional brazing material is used to secure the distal end of third needle subunit 240 to the external surface of second needle subunit 230, and additional brazing material is used to secure the distal end of second needle subunit 230 to the external surface of first needle subunit 220.

In some embodiments, the needle assembly is additionally or alternatively secured to the horn at a point inside the horn within or beyond the tip.

Laminated needle assembly 110 has a length of about 2 inches defined as the distance from the distal end of horn 210, or tip 310, to the distal end of first needle subunit 220. In some embodiments, the length of the needle assembly is between about 1 inch and about 4 inches, between about 1.5 inches and about 3 inches, between about 1.8 inches and about 2.3 inches, or between about 1.95 inches and about 2.15 inches. In some embodiments, the length of the needle assembly is at least about 1.5 inches, at least about 2 inches, or at least about 2.5 inches. In some embodiments, the length is about 2.04 inches or about 2.044 inches.

First needle subunit 220 has a gauge of about 18. In some embodiments, the first needle subunit has a gauge of between about 12 and about 24, between about 14 and about 22, or between about 16 and about 20. In some embodiments, the gauge of the first needle subunit is less than about 16, less than about 18, or less than about 20.

First needle subunit 220 has an inner diameter of about 0.03 inches. In some embodiments, the first needle subunit has an inner diameter of between about 0.01 inches and about 0.08 inches, between about 0.02 inches and about 0.05 inches, or between about 0.03 inches and about 0.04 inches. In some embodiments, the inner diameter of the first needle subunit is at least about 0.02 inches, at least about 0.03 inches, or at least about 0.04 inches.

Second needle subunit 230 has a length of about 1.475 inches defined as the distance between the distal and proximal ends. In some embodiments, the length of the second needle subunit is between about 0.5 inches and about 3 inches, between about 1 inch and about 2.5 inches, between about 1.4 inches and about 2 inches, or between about 1.43 inches and about 1.52 inches. In some embodiments, the length of the needle assembly is at least about 1 inch, at least about 1.3 inches, at least about 1.5 inches, or at least about 1.75 inches.

Second needle subunit 230 has a gauge of about 16. In some embodiments, the second needle subunit has a gauge of between about 10 and about 22, between about 12 and about 20, or between about 14 and about 18. In some embodiments, the gauge of the second needle subunit is less than about 14, less than about 16, or less than about 18.

Second needle subunit 230 has an inner diameter of about 0.04 inches. In some embodiments, the second needle subunit has an inner diameter of between about 0.02 inches and about 0.06 inches, between about 0.03 inches and about 0.05 inches, or between about 0.035 inches and about 0.045 inches. In some embodiments, the inner diameter of the second needle subunit is at least about 0.03 inches, at least about 0.04 inches, or at least about 0.045 inches.

Third needle subunit 240 has a length of about 0.734 inches defined as the distance between the distal and proximal ends. In some embodiments, the length of the second needle subunit is between about 0.3 inches and about 1.5 inches, between about 0.5 inches and about 1 inch, between about 0.7 inches and about 0.8 inches, or between about 0.71 inches and about 0.78 inches. In some embodiments, the length of the needle assembly is at least about 0.6 inches, at least about 0.8 inches, or at least about 1 inch.

Third needle subunit 240 has a gauge of about 14. In some embodiments, the third needle subunit has a gauge of between about 8 and about 20, between about 10 and about 18, or between about 12 and about 16. In some embodiments, the gauge of the third needle subunit is less than about 12, less than about 14, or less than about 16.

Third needle subunit 240 has an inner diameter of about 0.06 inches. In some embodiments, the third needle subunit has an inner diameter of between about 0.03 inches and about 0.09 inches, between about 0.04 inches and about 0.08 inches, or between about 0.05 inches and about 0.07 inches. In some embodiments, the inner diameter of the third needle subunit is at least about 0.05 inches, at least about 0.06 inches, or at least about 0.07 inches.

In some embodiments, at least one needle subunit comprises a thin-walled hypodermic tube. In some embodiments, at least one needle subunit comprises a regular-walled hypodermic tube. In some embodiments, at least one needle subunit comprises a thick-walled hypodermic tube. In some embodiments, at least one needle subunit comprises a thin-walled hypodermic tube, and at least one needle subunit comprises a regular-walled hypodermic tube. In some embodiments, at least one needle subunit comprises a thin-walled hypodermic tube, and at least one needle subunit comprises a thick-walled hypodermic tube. In some embodiments, at least one needle subunit comprises a thick-walled hypodermic tube, and at least one needle subunit comprises a regular-walled hypodermic tube.

According to some embodiments, the first needle subunit has a length of between about 1.9 inches and about 2.15 inches and has a gauge between about 16 and about 20; the second needle subunit has a length of between about 1.3 inches and about 1.6 inches and a gauge of between about 14 and about 18; and the third needle subunit has a length of between about 0.5 inches and about 1 inch and a gauge of between about 12 and about 16.

According to some embodiments, the first needle subunit has a length of between about 2 inches and about 2.1 inches and has a gauge between about 17 and about 19; the second needle subunit has a length of between about 1.4 inches and about 1.55 inches and a gauge of between about 15 and about 17; and the third needle subunit has a length of between about 0.65 inches and about 0.8 inches and a gauge of between about 13 and about 15.

In some embodiments, the laminated needle assembly includes only a first and a second needle subunit as those subunits have been described herein. In some embodiments, the laminated needle assembly includes only a first and a third subunit as those subunits have been described herein.

In the embodiment illustrated in FIG. 3, the inner diameter of second needle subunit 230 substantially corresponds to the gauge, or outer diameter, of first needle subunit 220. This feature is also illustrated in FIG. 4, which is a cross-sectional view of needle assembly 110 taken along line 4-4.

Similarly, the inner diameter of third needle subunit 240 substantially corresponds to the gauge, or outer diameter, or second needle subunit 230. In some embodiments, most or all of the inner surface of each successive needle subunit contacts the outer surface of the relevant underlying needle subunit. In some embodiments, the inner surface of each successive needle subunit contacts the outer surface of the relevant underlying needle subunit at least at those points where the two subunits are secured to each other.

Laminated needle assembly 110 has a length to diameter ratio of about 62:1 where the length is defined as the distance between tip 310 and the distal end of first needle subunit 220, and the diameter is the outer diameter of first needle subunit 220. In some embodiments, the length to diameter ratio of the laminated needle assembly is from about 20:1 to about 150:1, from about 45:1 to about 100:1, or from about 55:1 to about 80:1. In some embodiments, the laminated needle assembly has a length to diameter ratio of at least about 25:1, at least about 50:1, at least about 60:1, or at least about 80:1.

When any non-coaxial force is applied to a needle attached to a horn, such as occurs in ultrasonic applications, the needle experiences the greatest effect of that force at the location of attachment with the horn. If the non-coaxial force is strong enough or after enough exposure to the non-coaxial force, the needle will fracture and break at the attachment location.

With the needle subunits secured to each other, such as at both their respective proximal and distal ends, a parallel beam structure results that provides a surprising amount of strength to the overall needle assembly. Without being tied to any particular theory, it is believed that the use of a plurality of needle subunits—where each needle subunit is properly secured to the other subunits—distributes the effects of a non-coaxial force along that length of the first needle subunit rather than focusing those effects only at the location where the first needle assembly is attached to the horn. In other words, the first needle subunit experiences a fraction of the non-coaxial force at a location near the distal end of the second needle subunit. Another fraction of the force is experienced at a location near the distal end of the third needle subunit. Yet another fraction of the force is experienced at the tip of the horn where the needle assembly is secured to the horn.

And in those embodiments where the proximal ends of the second and subsequent needle subunits are secured to the tip of the horn, the combined thickness of the subunits allows for the resistance of a much greater non-coaxial force and/or a longer duration of such a force.

The laminated needle assemblies of the present disclosure may be manufactured using any suitable method. In some embodiments, a plurality of needle subunits are secured to each other using at least one of a brazing process, a welding process, a crimping process, or through a friction fit design.

In some embodiments, a brazing process is used to secure the proximal end and/or the distal end of each successive needle subunit to the underlying needle subunit. For example, in some methods, a first needle subunit having a desired length to diameter ratio is selected. A second needle subunit is selected that has an inner diameter corresponding to the outer diameter of the first needle subunit. A brazing process is used to secure the proximal end and/or the distal end of the second needle subunit to the outer surface of the first needle subunit. If desired, third, fourth, fifth, etc. needle subunits are selected having inner diameters that correspond to the outer diameters of the second, third, etc. needle subunits. Again, a brazing process is used to secure the proximal ends and/or the distal ends of the third, fourth, etc. needle subunits to the outer surfaces of the second, third, etc. needle subunits.

In some embodiments, a brazing process is used to secure the distal end of each successive needle subunit to the surface of the underlying needle subunit. Then, a brazing process is used to secure the proximal end of all the subunits to a horn or the tip of a horn.

In some embodiments, a brazing process is used to secure the plurality of needle subunits at one or more locations other than the proximal and distal ends. For example, in some embodiments, a brazing process is used to secure a portion of the first needle subunit at a position inside the horn beyond the tip. In some embodiments, a brazing process may be used to secure at least one of the plurality of needle subunits at a point between the distal and proximal ends of the relevant subunit.

Suitable brazing materials that may be used according to the present disclosure include aluminum-silicon, copper, copper-silver, copper-zinc, gold-silver, nickel alloy, silver, amorphous brazing foil using nickel, iron, copper, silicon, boron, phosphorus, or similar material, or a combination thereof.

According to some embodiments where brazing is used, the brazing temperature is selected as one that is below the annealing temperature of the material used for the needle subunits. In some embodiments, if the temperature is too high, the brazing process may additionally anneal at least a portion of the needle, thereby weakening the needle such that it will be less likely to withstand the forces and stress inherent in the use of ultrasonic energy.

In some embodiments, a brazing process is performed at a temperature below about 1,200° C., below about 1,000° C., below about 700° C., below about 500° C., below about 350° C., or below about 300° C. In some embodiments, a brazing process is performed for no more than about 60 seconds, no more than about 30 seconds, no more than about 10 seconds, no more than about 8 seconds, or no more than about 5 seconds. According to some embodiments, a brazing process is performed at a temperature of between about 500° C. and about 700° C. and for a duration of between about 2 seconds and about 8 seconds.

In some embodiments, the purpose of the brazing process is to cause a preformed brazing material to flow into the junction between horn 210 and laminated needle assembly 110 located at tip 310. To achieve this, the material is heated by heating horn 210 to an adequate temperature and kept at that temperature for as long as it takes for the material to flow into the juncture. In some cases, the temperature is between about 580° C. and about 600° C., and the duration is about 3 to about 7 seconds.

In some instances, too much heat can cause at least part of the laminated needle assembly to anneal, thereby reducing its strength. Thus, the brazing process is carefully controlled (for example, by limiting the temperature and/or the duration) so as to limit the amount of heat energy transferred to the laminated needle assembly from either the horn or the brazing material.

In some embodiments, using a brazing process according to the present disclosure achieves a unitary, continuous, and/or homogenous structure. Without being tied to any particular theory, it is believed that a homogenous structure acts as a better conduit for ultrasonic energy transferred from an ultrasonic delivery device to a target site or a target tissue.

In some embodiments, a crimping process is used to secure the proximal end and/or the distal end of each successive needle subunit to the underlying needle subunit. A crimping process is able to secure the plurality of needle subunits to each other by means of a friction fit or interference fit between the respective components. For example, in some methods, a first needle subunit having a desired length to diameter ratio is selected. A second needle subunit is mechanically formed by crimping. The second needle subunit is then able to be secured to the first needle subunit using a friction fit. If desired, third, fourth, fifth, etc. needle subunits are similarly mechanically formed using a crimping technique so as to provide for a friction fit with the second, third, etc. needle subunits. Once the needle assembly is complete, the horn can be crimped around the proximal end of the assembly to create an interference fit.

In some embodiments, a welding process is used to secure the proximal end and/or the distal end of each successive needle subunit to the underlying needle subunit. Suitable welding techniques can include electron beam welding, laser welding, and gas metal arc (GMAW) welding—such as metal inert gas (MIG) welding and metal active gas (MAG) welding.

For example, in some methods, a first needle subunit having a desired length to diameter ratio is selected. A second needle subunit is selected that has an inner diameter corresponding to the outer diameter of the first needle subunit. A welding process is used to secure the proximal end and/or the distal end of the second needle subunit to the outer surface of the first needle subunit. If desired, third, fourth, fifth, etc. needle subunits are selected having inner diameters that correspond to the outer diameters of the second, third, etc. needle subunits. Again, a welding process is used to secure the proximal ends and/or the distal ends of the third, fourth, etc. needle subunits to the outer surfaces of the second, third, etc. needle subunits.

In some embodiments, a welding process is used to secure the distal end of each successive needle subunit to the surface of the underlying needle subunit. Then, a welding process is used to secure the proximal end of all the subunits to a horn or the tip of a horn.

The laminated needle assemblies disclosed herein may be used for any suitable purpose where strength is desired but a relatively narrow needle is required. For example, the laminated needle assemblies are suitable for the delivery of ultrasonic energy, particular where the target of the energy requires a longer needle and/or the target is relatively hard so as to pose a greater structural threat to the needle. In the medical context, such targets can include musculoskeletal tissues such as bone.

According to some embodiments, the laminated needle assemblies can be used in cannula applications. In such applications, the laminated needle would add to the strength of the needle allowing for greater mobility and articulation in a minimally invasive surgery sight. Such would be the case in urology, plastic surgery, and proctology.

According to some embodiments, the laminated needle assemblies are used for ultrasonic mixing. In some instances an ultrasonic needle used for mixing compounds is needed to access a hard to reach area. The laminated needle design allows for transmission of ultrasonic power to a difficult-to-reach area to mix a material located in that area.

### Embodiments

Embodiment 1: A laminated needle assembly for delivering ultrasonic energy, the laminated needle assembly comprising: a first elongate portion defining a first length, the first elongate portion comprising: an inner diameter, an outer diameter, a proximal end, and a distal end; and a second elongate portion defining a second length and coaxially aligned with the first elongate portion, the second elongate portion comprising: an inner diameter, an outer diameter, a proximal end, and a distal end; wherein the first length of the first elongate portion is greater than the second length of the second elongate portion, wherein the inner diameter of the second elongate portion is no less than the outer diameter of the first elongate portion, the second elongate portion configured to surround at least a portion of an outer surface of the first elongate portion, and wherein the second elongate portion is secured to the first elongate portion at at least one of the distal end and the proximal end of the second elongate portion.

Embodiment 2: The laminated needle assembly of Embodiment 1, wherein the second elongate portion is secured to the first elongate portion at both the proximal and distal ends of the second elongate portion.

Embodiment 3: The laminated needle assembly of Embodiment 1, wherein the proximal end of the second elongate portion is secured to the first elongate portion at a location distal to the proximal end of the first elongate portion.

Embodiment 4: The laminated needle assembly of Embodiment 1, wherein the second elongate portion is secured to the first elongate portion by at least one of a brazing process, a crimping process and a welding process.

Embodiment 5: The laminated needle assembly of Embodiment 1, further comprising a third elongate portion coaxially aligned with both the first and second elongate portions, the third elongate portion having a proximal end and a distal end and defining a third length that is less than the second length, wherein the third elongate portion is secured to the second elongate portion at at least one of the proximal and distal ends of the third elongate portion.

Embodiment 6: The laminated needle assembly of Embodiment 4, wherein the third elongate portion is secured to the second elongate portion at both the proximal end and the distal end of the third elongate portion.

Embodiment 7: The laminated needle assembly of Embodiment 4, wherein the proximal end of the third elongate portion is substantially aligned with the proximal end of the second elongate portion.

Embodiment 8: The laminated needle assembly of Embodiment 4, wherein the third elongate portion is secured to the second elongate portion by at least one of a brazing process, a crimping process, and a welding process.

Embodiment 9: The laminated needle assembly of Embodiment 1, wherein the length of the first elongate portion is between about 1.5 inches and about 2.5 inches.

Embodiment 10: The laminated needle assembly of Embodiment 1, wherein the length of the first elongate portion is between about 1.8 inches and about 2.2 inches.

Embodiment 11: The laminated needle assembly of Embodiment 1, wherein the length of the first elongate portion is between about 1.95 inches and about 2.1 inches.

Embodiment 12: The laminated needle assembly of Embodiment 1, wherein a ratio between the length of the first elongate portion and the outer diameter of the first elongate portion is between about 20:1 and about 100:1.

Embodiment 13: The laminated needle assembly of Embodiment 1, wherein a ratio between the length of the first elongate portion and the outer diameter of the first elongate portion is at least about 25:1.

Embodiment 14: The laminated needle assembly of Embodiment 1, wherein a ratio between the length of the first elongate portion and the outer diameter of the first elongate portion is at least about 50:1.

Embodiment 15: The laminated needle assembly of Embodiment 1, wherein a ratio between the length of the first elongate portion and the outer diameter of the first elongate portion is between about 50:1 and about 75:1.

Embodiment 16: A method of using the laminated needle assembly of Embodiment 1 to deliver ultrasonic energy, wherein the ultrasonic energy is delivered as a part of one of the following medical procedures: urology, plastic surgery, proctology and wound debridement.

Embodiment 17: A method of using the laminated needle assembly of Embodiment 1 to deliver ultrasonic energy to mix one or more compounds in a difficult-to-access region of the human body.

Embodiment 18: A method of manufacturing a laminated needle assembly, the method comprising: securing a first elongate portion to a second elongate portion—the first elongate portion defining a first length and comprising: an inner diameter, an outer diameter, a proximal end, and a distal end—the second elongate portion defining a second length and coaxially aligned with the first elongate portion and comprising: an inner diameter, an outer diameter, a proximal end, and a distal end; wherein the first length of the first elongate portion is greater than the second length of the second elongate portion, wherein the inner diameter of the second elongate portion is no less than the outer diameter of the first elongate portion, the second elongate portion configured to surround at least a portion of an outer surface of the first elongate portion, and wherein the second elongate portion is secured to the first elongate portion at at least one of the distal end and the proximal end of the second elongate portion.

Embodiment 19: The method of Embodiment 18, wherein securing the first and second elongate portions comprises securing the first elongate portion at both the proximal end and the distal end of the second elongate portion.

Embodiment 20: The method of Embodiment 18, further comprising, prior to securing the first and second elongate portions, substantially aligning the proximal end of the second elongate portion with the proximal end of the first elongate portion.

Embodiment 21: The method of Embodiment 18, wherein securing the first and second elongate portions comprises the use of at least one of a brazing process, a crimping process, and a welding process.

Embodiment 22: The method of Embodiment 18, further comprising securing a third elongate portion to the second and first elongate portions, the third elongate portion comprising: a proximal end, a distal end, and a third length that is less than the second length.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the embodiments of the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements. In one embodiment, the terms “about” and “approximately” refer to numerical parameters within 10% of the indicated range.

The terms “a,” “an,” “the” and similar referents used in the context of describing the embodiments of the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the embodiments of the present disclosure and does not pose a limitation on the scope of the present disclosure. No language in the specification should be construed as indicating any non-claimed element essential to the practice of the embodiments of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventor for carrying out the embodiments of the present disclosure. Of course, variations on these described embodiments will become apparent to those of ordinary skill in the art upon reading the foregoing description. The inventor expects skilled artisans to employ such variations as appropriate, and the inventor intends for the embodiments of the present disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of this disclosure so claimed are inherently or expressly described and enabled herein.

In closing, it is to be understood that the embodiments disclosed herein are illustrative of the principles of the present disclosure. Other modifications that may be employed are within the scope of this disclosure. Thus, by way of example, but not of limitation, alternative configurations of the embodiments of the present disclosure may be utilized in accordance with the teachings herein. Accordingly, the present disclosure is not limited to that precisely as shown and described.

1. A laminated needle assembly for delivering ultrasonic energy, the laminated needle assembly comprising:
a first elongate portion defining a first length, the first elongate portion comprising: an inner diameter, an outer diameter, a proximal end, and a distal end; and
a second elongate portion defining a second length and coaxially aligned with the first elongate portion, the second elongate portion comprising: an inner diameter, an outer diameter, a proximal end, and a distal end;
wherein the first length of the first elongate portion is greater than the second length of the second elongate portion,
wherein the inner diameter of the second elongate portion is no less than the outer diameter of the first elongate portion, the second elongate portion configured to surround at least a portion of an outer surface of the first elongate portion, and
wherein the second elongate portion is secured to the first elongate portion at at least one of the distal end and the proximal end of the second elongate portion.

2. The laminated needle assembly of claim 1, wherein the second elongate portion is secured to the first elongate portion at both the proximal and distal ends of the second elongate portion.

3. The laminated needle assembly of claim 1, wherein the proximal end of the second elongate portion is secured to the first elongate portion at a location distal to the proximal end of the first elongate portion.

4. The laminated needle assembly of claim 1, wherein the second elongate portion is secured to the first elongate portion by at least one of a brazing process, a crimping process and a welding process.

5. The laminated needle assembly of claim 1, further comprising a third elongate portion coaxially aligned with both the first and second elongate portions, the third elongate portion having a proximal end and a distal end and defining a third length that is less than the second length, wherein the third elongate portion is secured to the second elongate portion at at least one of the proximal and distal ends of the third elongate portion.

6. The laminated needle assembly of claim 4, wherein the third elongate portion is secured to the second elongate portion at both the proximal end and the distal end of the third elongate portion.

7. The laminated needle assembly of claim 4, wherein the proximal end of the third elongate portion is substantially aligned with the proximal end of the second elongate portion.

8. The laminated needle assembly of claim 4, wherein the third elongate portion is secured to the second elongate portion by at least one of a brazing process, a crimping process, and a welding process.

9. The laminated needle assembly of claim 1, wherein the length of the first elongate portion is between about 1.5 inches and about 2.5 inches.

10. The laminated needle assembly of claim 1, wherein the length of the first elongate portion is between about 1.8 inches and about 2.2 inches.

11. The laminated needle assembly of claim 1, wherein the length of the first elongate portion is between about 1.95 inches and about 2.1 inches.

12. The laminated needle assembly of claim 1, wherein a ratio between the length of the first elongate portion and the outer diameter of the first elongate portion is between about 20:1 and about 100:1.

13. The laminated needle assembly of claim 1, wherein a ratio between the length of the first elongate portion and the outer diameter of the first elongate portion is at least about 25:1.

14. The laminated needle assembly of claim 1, wherein a ratio between the length of the first elongate portion and the outer diameter of the first elongate portion is at least about 50:1.

15. The laminated needle assembly of claim 1, wherein a ratio between the length of the first elongate portion and the outer diameter of the first elongate portion is between about 50:1 and about 75:1.

16. A method of using the laminated needle assembly of claim 1 to deliver ultrasonic energy, wherein the ultrasonic energy is delivered as a part of one of the following medical procedures: urology, plastic surgery, proctology, and wound debridement.

17. A method of using the laminated needle assembly of claim 1 to deliver ultrasonic energy to mix one or more compounds in a difficult-to-access region of the human body.

18. A method of manufacturing a laminated needle assembly, the method comprising:
securing a first elongate portion to a second elongate portion—the first elongate portion defining a first length and comprising: an inner diameter, an outer diameter, a proximal end, and a distal end—the second elongate portion defining a second length and coaxially aligned with the first elongate portion and comprising: an inner diameter, an outer diameter, a proximal end, and a distal end;
wherein the first length of the first elongate portion is greater than the second length of the second elongate portion,
wherein the inner diameter of the second elongate portion is no less than the outer diameter of the first elongate portion, the second elongate portion configured to surround at least a portion of an outer surface of the first elongate portion, and
wherein the second elongate portion is secured to the first elongate portion at at least one of the distal end and the proximal end of the second elongate portion.

19. The method of claim 18, wherein securing the first and second elongate portions comprises securing the first elongate portion at both the proximal end and the distal end of the second elongate portion.

20. The method of claim 18, further comprising, prior to securing the first and second elongate portions, substantially aligning the proximal end of the second elongate portion with the proximal end of the first elongate portion.

21. The method of claim 18, wherein securing the first and second elongate portions comprises the use of at least one of a brazing process, a crimping process, and a welding process.

22. The method of claim 18, further comprising securing a third elongate portion to the second and first elongate portions, the third elongate portion comprising: a proximal end, a distal end, and a third length that is less than the second length.
